(19) United States
(12) Patent Application Publication
(10) Pub. No.: US 2004/0220600 A1
(43) Pub. Date: Nov. 4, 2004

(54) Surgical knives

(76) Inventors: Terence Arnold Waldock, Bedfordshire (GB); James C Hays, Atlanta, GA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/482,197

(22) PCT Filed: Jun. 28, 2002
(86) PCT No.: PCT/GB02/02998

(30) Foreign Application Priority Data
Jun. 28, 2001 (GB) 0115747.8

(51) Int. Cl. B26B 19/28; A61B 17/32

(52) U.S. Cl. 606/167; 30/42

(57) ABSTRACT

A knife for ophthalmic surgery has a diamond blade (14) which protrudes at the forward end of a handle (11) through a slot in a footplate (16). The surface of the footplate which contracts the tissue (cornea or sclera, for example) to be incised has irregular castellations (28a, 28b, 28c, 28d, 28e, 28a′, 28b′, 28c′, 28d′, 28e′) forming irregular grooves (32a, 32b, 34a, 34b, 32a′, 32b′, 34a′, 34b′) therebetween. Alternatively or in addition, the contact surface has protruding pins (140) which engage the tissue (sclera, usually) to be incised.

This invention relates to knives used in ophthalmic surgery, and is particularly concerned with knives for use where incisions are made in the cornea or in the sclera. Such knives commonly have a blade formed from diamond.

Diamond knives for ophthalmic surgery are known where the leading end of the knife has a pivotable footplate which rests on the surface to be cut and through which the diamond blade projects.

Our UK patent specification GB-B-2305608 describes surgical knives of this type where the end of a pivotable footplate which is placed in contact with the surface of the tissue to be incised is provided with castellations, indentations, grooves or roughness, so that there is a reduced tendency for the footplate to slip relative to the tissue as an incision is made.

In the examples illustrated in the aforesaid patent specification the contact surface of the pivotable footplate is shown with regular, equispaced indentations or castellations, each having the same shape.

It has now been found, in accordance with a first aspect of the present invention, that an improved performance can be achieved if the contact surface configuration of the pivotable footplate is not regular.

The invention accordingly provides a surgical knife comprising a handle, a cutting blade at the forward end of the knife, and an integral footplate at the forward end of the knife through which the blade can protrude, wherein

the footplate has at least one contact surface to be located on tissue to be incised,

the handle and blade are jointly pivotally movable relative to the footplate so that with said footplate surface anchored on the tissue a pivoting movement of the handle will cause the blade to traverse a cutting path across the tissue, and

the said contact surface of the footplate has a configuration which is not regular.

This is conveniently achieved if the contact surface has castellations of which some are smaller than others, thereby to create surface grooves of which some are larger than others.

In this way, more tissue is able to enter the grooves, between the castellations, as compared with a regular array. There is also better fixation of the footplate on the tissue, and a reduced risk of the footplate sliding on the tissue.

Preferably, at least some of the castellations are tapered, with a relatively narrow contact surface tapering to a larger foot, the castellations being, for example, trapezoidal in cross-sectional shape. In this way, one can have alternate larger and smaller grooves along the length of the contact surface.

Preferably, the surface configuration comprises irregular castellations forming grooves therebetween.

The grooves may also be irregular.

The constellations and/or the grooves may be irregular in at least one of their respective shapes, sizes and spacings.

In a preferred arrangement, the constellations form a set of alternate ones of constant cross-section and ones of diminishing cross-section, the set extending in the direction of movement of the blade in relation to the footplate.

Advantageously, the blade moves in a slot in the footplate, the slot extending in the direction of movement of the blade and dividing the said contact surface into first and second surface portions lying to respective opposite sides of the slot.

In such an arrangement, the castellations on the respective surface portions are preferably aligned across the slot so that the grooves in the respective surface portions are also aligned.

The invention also provides, in a second aspect, a surgical knife comprising a handle, a cutting blade at the forward end of the knife, and an integral footplate at the forward end of the knife through which the blade can protrude, wherein

the footplate has at least one contact surface to be located on tissue to be incised,

the handle and blade are jointly pivotally movable relative to the footplate so that with said footplate surface anchored on the tissue a pivoting movement of the handle will cause the blade to traverse a cutting path across the tissue, and

the said contact surface has a plurality of spaced-apart protruding pins for engagement with tissue to be incised.

Preferably, the pins located at respective corners of the footplate.

The footplate is preferably rectangular, which term includes square.

It is again advantageous that the blade moves in a slot in the footplate, the slot extending in the direction of movement of the blade and dividing the said contact surface into first and second surface portions lying to respective opposite sides of the slot.

Advantageously, at least some of the pins surmount castellations on the said contact surface.

Although the improved pivotable footplate of knives of both aspects of the present invention is particularly well-suited to use in a step knife, i.e. a knife with a number of predetermined depth-of-cut settings, it can also be used with knives having a micrometer adjustment.

In a knife according to either aspect of the invention, it is preferred that the pivoting movement of the handle relative to the footplate causes the blade cutting depth to change as the cutting path is traversed. Alternatively, the cutting depth can be constant over the length of the cutting path.

Preferably, the depth of cut increases by of the order of 0.035 mm over a path length of the order of 3 mm.

Preferably, the depth of cut increases from of the order of 0.6 mm to of the order of 0.635 mm.

In a knife according to either aspect of the invention, the footplate is preferably substantially U-shaped with a pair of rearwardly extending limbs connected pivotally to the handle, and with stop means to limit the relative pivotal movement.

In order that the invention may be more fully understood, two embodiments of the invention will now be described by way of example and with reference to the accompanying drawings, in which:

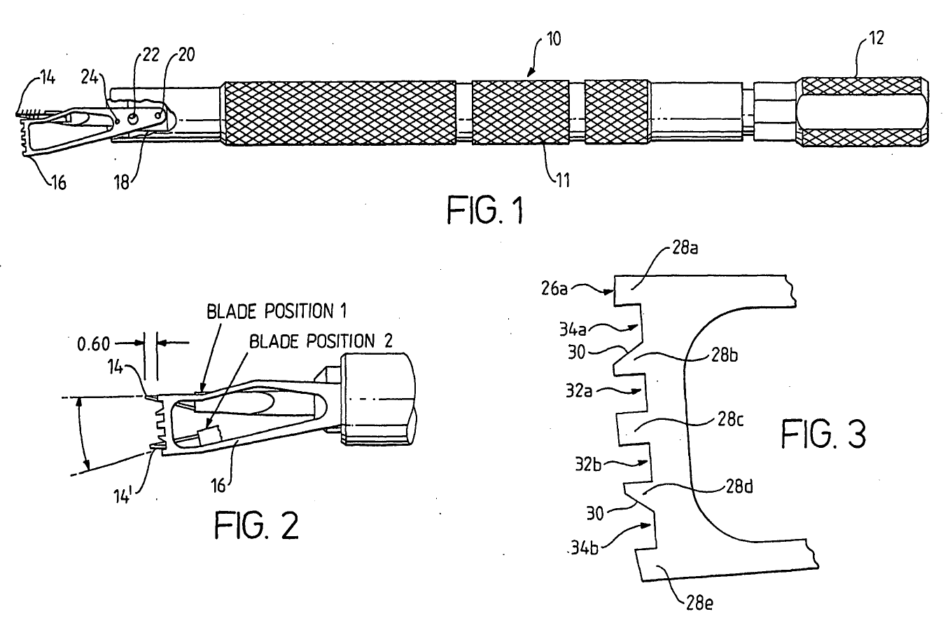

FIG. 1 shows a diamond step knife for use in ophthalmic surgery;

FIG. 2 is a side view of the tip of the knife of FIG. 1, shown on an enlarged scale;

FIG. 3 is a side view of the front end of the tip of the knife of FIGS. 1 and 2, shown on a further enlarged scale;

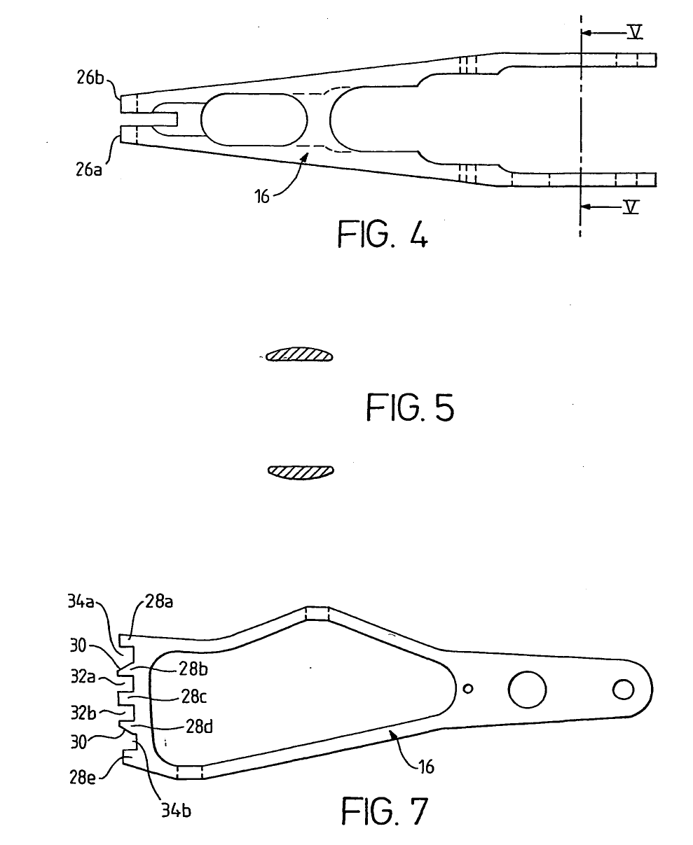

FIG. 4 is a top view of a pivotable footplate of the knife of FIG. 1, shown on an enlarged scale;

FIG. 5 is a section on the line V-V on FIG. 4;

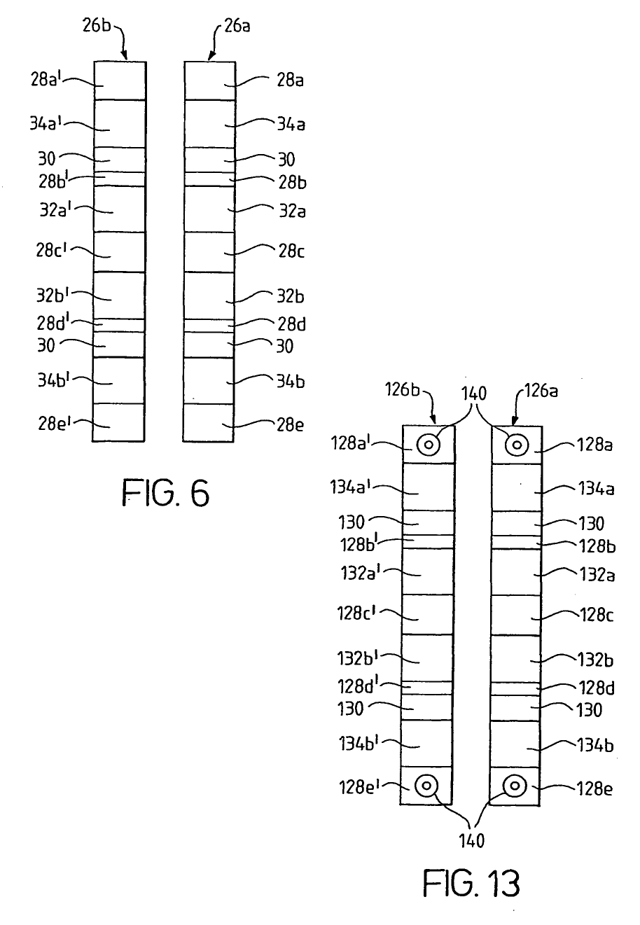

FIG. 6 is an end view of the footplate of FIG. 4, enlarged even more;

FIG. 7 is a side view of the footplate of FIG. 4;

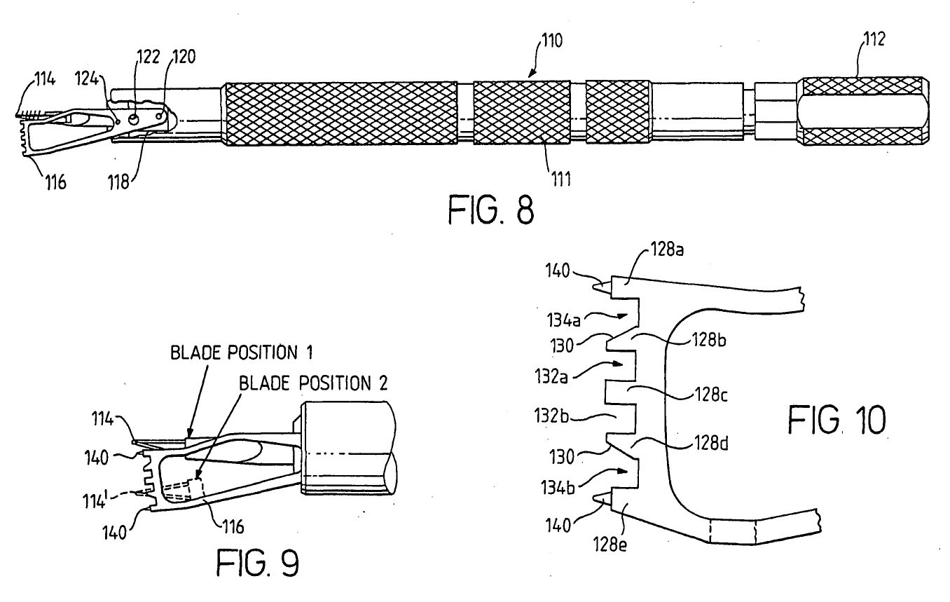

FIG. 8 is a side view of another diamond step knife;

FIG. 9 is a side view of the tip of the knife of FIG. 8, shown on an enlarged scale;

FIG. 10 is side view of the front end of the tip of the knife of FIGS. 8 and 9, shown on a further enlarged scale;

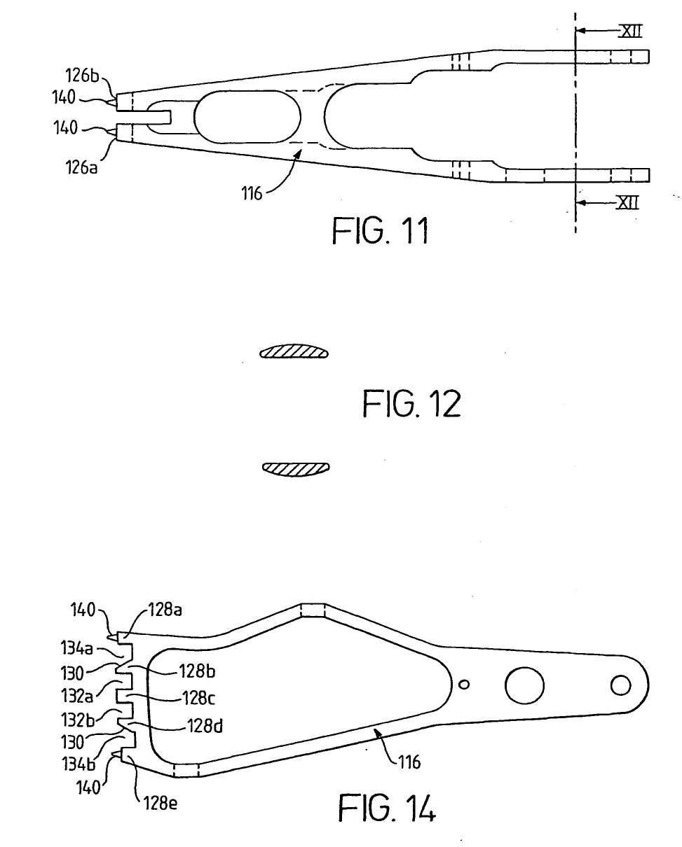

FIG. 11 is a top view of a pivotable footplate of the knife of FIG. 8, shown on an enlarged scale;

FIG. 12 is a section on the line XII-XII of FIG. 11;

FIG. 13 is an end view of the footplate of FIG. 11, enlarged even more; and

FIG. 14 is a side view of the footplate of FIG. 11.

FIG. 1 shows a step knife 10 having a knurled handle 11 and a setting mechanism 12 at the rearward end of the handle to enable a diamond blade 14 which protrudes from the forward end of the knife to be set for a chosen one of a number of predetermined cutting depth settings, for example 0.60 mm. Apart from the modifications herein described, the knife of FIGS. 1 to 7 corresponds closely to that of GB-B-2305608, to which reference should be made if necessary and the entire contents of which are incorporated herein by reference.

A generally U-shaped footplate 16 is mounted at the forward end of the knife and is set for pivoting movement relative to a guard pivot 18. Pivoting takes place about a air of taper pins 20, 22. A stop pin is shown at 24. The front end face of the footplate is in fact two spaced parallel faces 26a, 26b (see FIG. 6) with the blade 14 (not shown in FIG. 6) between them. The end face is slightly curved and is indented or castellated in an irregular configuration. Five castellations 28a, 28b, 28c, 28d and 28e are provided in the end face 26a; five more 28a′, 28b′, 28c′, 28d′ and 28e′ are provided in the end face 26b. Castellations 28a, 28c, 28e, 28a′, 28c′ and 28e′ are each substantially square in cross-section. The intermediate castellations 28b′ and 28d′ on the end face 26a each have a tapering-side face 30¹ to make them each substantially trapezoidal in cross-section. This provides two central grooves 32a, 32b which are of the same size and two outer grooves 34a, 34b which are of equal size but larger. The intermediate castellations 28b′ and 28d′ on the end face 26b each have a tapering side face 30′ to make then each substantially trapezoidal in cross-section. This provides two central grooves 32a′, 32b′ which are of the same size and two outer grooves 34a′, 34b′ which are of equal size but larger. It will be noted that the castellations and grooves on one end face 26a are aligned with the castellations and grooves on the other end face 26b across the gap between the end faces 26a, 26b. The arrangement of irregular castellations enables more tissue to be received in the grooves, helping to improve fixation and avoiding slip of the footplate. As indicated on FIG. 3, the length dimension of the front end of each of castellations 28b and 28d is 0.14 mm. The arrangement of castellations is also shown in FIGS. 6 and 7. FIGS. 4 and 5, together with FIGS. 6 and 7 show the pivotable footplate 16 in more detail.

FIG. 2 shows the blade 14 in each of its two extreme positions 14, 14′ in relation to the footplate. In each blade position the blade depth is 0.60 mm. The arc of movement of the blade is 3.6 mm. The blade cannot stop at any intermediate point between the two limit positions.

The knife is used as described in GB-B-2305608, the footplate 16 being placed in contact with the tissue to be incised, for example the cornea, and the handle 11 and blade 14 simply pivoted relative to the footplate. This pivoting movement causes the blade 14 to traverse the footplate 16 in an arcuate path and to cut into the tissue at a constant depth. In a modification, the footplate could be shaped so that the depth of cut increases along the length of the cut, for example by 0.035 mm. This is preferable when the cornea is to be incised; a constant depth is preferable if the sclera is to be incised.

Although in the embodiment described above five castellations are provided on each end face 28b, 28b′, the invention is not to be regarded as limited to this number. Also, the shape of the individual castellations may differ from that shown.

FIGS. 8 to 14 of the drawings show a second knife and correspond to FIGS. 1 to 7, respectively. Parts corresponding to those of the first knife are indicated by reference numerals increased by “100”.

The knife of FIGS. 8 to 14 differs from that of FIGS. 1 to 7 in that each of the four castellations 128a, 128e, 128a′ and 128e′ is provided with a pin 140 which is conical in shape and has a circular base of 0.215 mm in diameter, an axial length of 0.30 mm and a tip diameter of 0.05 mm. In all other respects the knife of FIGS. 8 to 14 corresponds exactly to that of FIGS. 1 to 7, to the above description of which reference should be made, if necessary along with reference to GB-B-2305608 the entire contents of which are incorporated herein by reference. The cutting depth may be constant or increasing, as described above.

In use of the knife, the pins 140 engage the tissue in which an incision is to be made. This helps to prevent the footplate sliding relative to the surface of the tissue and stabilises the position of the knife relative to the tissue. Whilst this engagement would be undesirable if the tissue were the cornea, it is perfectly acceptable when the tissue is the sclera which, being essentially muscle, will simply re-grow afterwards. Any damage caused by the pins will therefore heal.

Although in the embodiment described above four pins are provided, the invention is not to be regarded as limited to this number. Also, the shape of the pins may differ from that shown, with pins of, for example, square cross-section also being possible.

Although the invention has been described in both aspects in relation to a step knife, it can also be used in both aspects with knives having a micrometer setting mechanism, in which knives where the blade can be arranged to cut to a constant or an increasing depth as the blade traverses from one end of the footplate to the other, such as are described in GB-B-2305608.

1. A surgical knife comprising a handle, a cutting blade at a forward end of the knife, and an integral footplate at the forward end of the knife, through which the blade can protrude, wherein the footplate has at least one footplate contact surface to be located on a tissue to be incised, the handle and blade being jointly pivotally movable relative to the footplate so that with said footplate contact surface anchored on the tissue, a pivoting movement of the handle causes the blade to traverse a cutting path across the tissue; the footplate contact surface having a configuration which is not regular.

2. A surgical knife according to claim 1, in which the surface configuration comprises irregular castellations forming grooves therebetween.

3. A surgical knife according to claim 2, in which the grooves are also irregular.

4. A surgical knife according to claim 2, in which the castellations are irregular in at least one of their shape, their size, and their spacing.

5. A surgical knife according to claim 3, in which the grooves are irregular in at least one of their shape, their size, and their spacing.

6. A surgical knife according to claim 2, in which the castellations form a set of alternating constant cross-section castellations and diminishing cross-section castellations, the set extending in the direction of movement of the blade relative to the footplate.

7. A surgical knife according to claim 1, in which the blade moves in a slot in the footplate, the slot extending in the direction of movement of the blade and dividing the footplate contact surface into first and second surface portions lying to respective opposite sides of the slot.

8. A surgical knife according to claim 7, in which the castellations on the respective surface portions are aligned across the slot so that the grooves in the respective surface portions are also aligned.

9. A surgical knife comprising a handle, a cutting blade at a forward end of the knife; and an integral footplate at the forward end of the knife through which the blade can protrude, wherein the footplate has at least one contact surface to be located on tissue to be incised, the handle and blade jointly pivotally movable relative to the footplate so that with said footplate contact surface anchored on the tissue, a pivoting movement of the handle causes the blade to traverse a cutting path across the tissue, the footplate contact surface having a plurality of spaced-apart protruding pins for engagement with tissue to be incised.

10. A surgical knife according to claim 9, in which the pins comprise pins located at respective corners of the footplate.

11. A surgical knife according to claim 10, in which the footplate is rectangular.

12. A surgical knife according to claim 9, in which the blade moves in a slot in the footplate, the slot extending in the direction of movement of the blade and dividing the footplate contact surface into first and second surface portions lying to respective opposite sides of the slot.

13. A surgical knife according to claim 9, in which at least some of the pins surmount castellations on the contact surface.

14. A surgical knife according to a claim 1, in which the pivoting movement of the handle relative to the footplate causes the blade cutting depth to change as the cutting path is traversed.

15. A surgical knife according to claim 14, in which the cutting depth increases by about 0.035 mm over a path length of about 3 mm.

16. A surgical knife according to claim 15, in which the cutting depth increases from about 0.6 mm to about 0.635 mm.

17. A surgical knife according to claim 1, in which the footplate is substantially U-shaped with a pair of rearwardly extending limbs connected pivotally to the handle, with stop means to limit the relative pivotal movement.

18. A surgical knife according to claim 1, in which the handle incorporates a micrometer setting mechanism.

19. (Canceled)
